(19) United States
(12) Patent Application Publication
(10) Pub. No.: US 2025/0032380 A1
(43) Pub. Date: Jan. 30, 2025

(54) Cosmetic Composition For Restructuring Hair And Improving The Appearance Thereof

(71) Applicant: GIULIANI S.P.A., Milano (IT)

(72) Inventors: Giammaria GIULIANI, Montagnola (CH); Fabio RINALDI, Milano (IT); Daniela PINTO, Milano (IT); Antonio MASCOLO, Milano (IT); Barbara MARZANI, Carbonara Al Ticino (IT)

(21) Appl. No.: 18/718,625

(22) PCT Filed: Dec. 14, 2022
(86) PCT No.: PCT/EP2022/085950

§ 371 Date: Jun. 11, 2024

(30) Foreign Application Priority Data
Dec. 15, 2021 (IT) 102021000031451

(51) Int. Cl. A61K 8/41; A61Q 5/00

(52) U.S. Cl. CPC A61K 8/41; A61Q 5/002

(57) ABSTRACT

The present invention relates to the cosmetic use of alkyl spermidine, in particular methylspermidine, by application of the same on hair shafts for restructuring the cuticle and/or improving the hair outward appearance. Preferably the alkylspermidines are formulated as a composition suitable for application to the hair shaft.

### FIELD OF THE INVENTION

The present invention concerns a cosmetic composition for restructuring hair and improving the outward appearance thereof.

The present invention originates in the sector of cosmetic products and in particular of products for hair care and treatment.

In particular, the present invention relates to a cosmetic preparation in the form of a hair mask to be applied along the hair shaft for restructuring the cuticles and improving the outward appearance thereof.

### PRIOR ART

Hair appearance and conditions play an important role in today's society as the outward appearance thereof helps determine the image and first impression that an individual conveys to others.

Hair appearance is related to the conditions of its complex structure formed by cuticle, cortex, medulla, and Cell Membrane Complex (CMC).

The cuticle represents the outer protective layer that covers the hair fiber and is made up of flattened, very thin, transparent keratinized cells without a nucleus that are arranged obliquely, overlapped like the tiles of a roof with the free edge facing the apex of the hair.

The cuticle represents the hair protective layer and constitutes the outermost layer of the same. There are approximately 6-10 layers of cuticles which repeat themselves to form a cuticle section, such layers are held together by the CMC cementing substance. Some types of CMC are known, depending on their location:
Cuticle-cuticle CMC which contains 18-MEA acid. The outermost layer of the cuticle (epicuticle or f-layer) is a protein layer coated with structural lipids (18-MEA acid) covalently bonded to it
CMC cuticle-cortex. It is responsible for the connection between the innermost cuticle layer and the outermost cortical layer. It has characteristics that are intermediate between the other two;
Cortex-cortex CMC. It is the layer that is interposed between cortical cells, in the two beta portions the lipids are free, i.e. non-covalently linked to the protein matrix.

The hair outward appearance is a consequence of the individual type of hair, i.e. shape and colour, and of other factors such as exposure to external agents, typically solar radiation and smog, and aesthetic treatments such as dyes and permanent.

The latter are chemical-type treatments that cause the hair structure to break, leading to curling of the hair which also gives it greater volume and softness.

An intact and well-ordered cuticle layer with aligned and well-overlapping scales favours light reflection, increasing brightness; in addition, it prevents or reduces hair porosity thus improving the texture thereof.

The friction forces that are applied while brushing the hair, alone, are sufficient to damage it.

Due to the delipidizing action of surfactants, frequent washing may also damage the cuticle layer and, following its partial removal, the cortical structures.

The removal of unbound lipids from the innermost layers is favoured by the chemicals present in shampoos.

The CMC, as well as some cuticle sub-units, represents one of the regions most vulnerable to treatments such as colouring/bleaching, styling, and straightening.

However, even treatments that are generally considered harmless for daily hair styling and cleaning, if carried out inappropriately, may cause damage to the hair outermost layer (f-layer) with appreciable consequences on brightness or overall beauty of the hair, and resulting in an increase in porosity, breakage or formation of split ends.

Permanent or straightening using chemical methods are methods that permanently change hair appearance by damaging the structure thereof.

In permanent hair treatment, the cystine disulfide bonds are initially “broken” by applying an alkaline medium containing a reducing substance (thioglycolate, bisulfites, etc.) with the consequent formation of a double number of cysteine residues. After having given the desired shape to the hair, an oxidizing substance is applied, typically hydrogen peroxide, to reconstitute the disulfide bridges and “seal” the new hair fold in the new positions. The alkaline medium makes the reducing substance permeate the hair innermost layers, up to the cortex, so that more bonds can be broken and then closed in the desired position.

Alkaline treatments in general, e.g. straightening, have a direct effect on the f-layer as they can cause the removal of 18-MEA molecules, which is the major component of the f-layer.

The damages of these treatments are reflected both at the level of lipids (decrease in content) in the cuticle CMC, and at the level of the cortex.

Due to their location, the hair, as well as the skin, are directly and constantly exposed to air, solar radiation, environmental pollutants, or mechanical and chemical insults which are capable of inducing the production of free radicals in the body.

Cosmetic treatments and exposure to external agents cause changes in some of the hair optical properties and interfere in the phenomena of light refraction, reflection, absorption and diffusion.

Furthermore, hair treatments are often the main culprits for hair brittleness, mainly due to the lifting of the hair cuticle, an operation which is necessary in most colouring, bleaching and permanent treatments. The lifting of the cuticle helps to make the hair brittle and susceptible to dryness, dullness and breakage, elements that interfere with the hair outward appearance.

Therefore, there is a need to have suitable treatments to improve the hair outward appearance, in particular when damaged by the action of external agents and by permanent and/or colouring treatments.

A general object of the present invention therefore consists in providing cosmetic products that, when applied to the hair shaft, improve its outward appearance.

Another object is to provide cosmetic products to be applied to the hair shaft containing cosmetic substances capable of restoring or preserving the integrity of the hair cuticle layer in order to improve the optical properties and consequently improve the outward appearance of the same.

### SUMMARY OF THE INVENTION

In the context of research activities on optical properties of the hair and its outward appearance, it was found that the alignment of multiple hair fibers, their cross-sectional shape and the state of the cuticle play an important role in determining the hair outward appearance.

In the context of research activities in the cosmetic and trichological sector, the Applicant has unexpectedly found that some selected molecules which play a role in the hair greying process, when applied to the hair shaft, perform a restructuring action on the cuticle which results in a noticeable improvement in the hair outward appearance.

This observation, from which the present invention originates, was the object of in-depth analysis using sophisticated optical and electron microscope observation techniques.

Starting from this observation, the inventors have surprisingly found that direct application of alkyl spermidines, in particular methylspermidine, on the hair surface, in particular on the keratin-based scales, reduces breakage points in the hair fibers and recompacts the external structure of the capillary cuticle increasing hair gloss, softness and aesthetic appearance.

According to a general aspect, the object of the present invention is therefore the cosmetic use of a compound of formula (I)

H₂N—(CH₂)₃—N¹(R)—(CH₂)₄—NH₂ (I)

wherein R is a substituent group linked to the secondary amino function of spermidine, selected from:
linear or branched, saturated alkyl groups comprising from 1 to 6 carbon atoms;
saturated cycloalkyl group
in the form of a free base or pharmaceutically acceptable salt thereof,
to improve the outward appearance of a keratin-based structure of an individual by applying it to at least a portion of said keratin structure.

According to some embodiments, the substituent group (R) is selected from methyl, ethyl, propyl, isopropyl, butyl, isobutyl, pentyl, hexyl.

Advantageously, the compounds of formula (I) or a composition containing them are applied to the hair shafts of an individual.

Advantageously, the cosmetic use of the compounds of formula (I) or a composition containing them described herein does not provide for their application to the scalp of an individual. According to these embodiments, the use or application of the compounds of formula (I) or compositions containing them to the scalp is therefore excluded.

When applied to the hair, the compounds of formula (I) recompact the external structure of the capillary cuticle.

Advantageously, the application of a compound of formula (I), or a cosmetic composition containing it, seals the breakage points of the hair (Trichorrhexis), areas in which a thickening is formed due to swelling, making the corneal surface scaly.

Advantageously, the cosmetic use of the compounds of formula (I) or cosmetic compositions containing them prevents or treats the lifting of the hair cuticle or keratin scales.

The hair treated with the method described herein appears glossy and shiny, and its structure is less vulnerable to aggression by external agents.

Furthermore, the cosmetic use according to the invention reduces the electrostatic phenomena and the structural damage of the keratin structures, such as hair.

According to another aspect, the invention relates to the cosmetic use of a cosmetic composition comprising a compound of formula (I)

H₂N—(CH₂)₃—N¹(R)—(CH₂)₄—NH₂ (I)

wherein R is a substituent group linked to the secondary amino function of spermidine, selected from:
linear or branched, saturated alkyl groups comprising from 1 to 6 carbon atoms;
saturated cycloalkyl group
   or a cosmetically acceptable salt thereof, and a cosmetically acceptable carrier as a restructuring agent for a keratin structure and/or as a cuticle repairer and/or for improving the outward appearance of an individual's keratin structure by applying a cosmetically acceptable amount of the same to the keratin structure, especially hair shafts or portions thereof.

Preferred compounds of formula (I) are those in which R is C₁-C₆, more preferably C₁-C₃.

Within the scope of the invention, the preferred compound of formula (I) is methyl spermidine or N-(3-aminopropyl)-N¹-methyl-1,4-Butanediamine having the following formula:

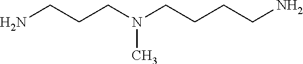

According to some aspects, the invention relates to the cosmetic use of a cosmetic composition as described herein as a hair restructuring agent and/or for repairing the cuticle.

Hair treated with the cosmetic composition has a glossy appearance, reflects light, is soft, easy to comb and has a silky appearance without being weighed down by the application of the composition.

### BRIEF DESCRIPTION OF THE DRAWINGS

The characteristics and advantages of the present invention will become more apparent from the enclosed drawings in which:

FIG. 1 shows optical microscope micrographs (b) and (d) and scanning electron microscope (SEM) micrographs (a) (c) of hair with substantially flat cuticles (a) and (b) and with uplifted cuticles (c), (d);

FIG. 2a shows images, acquired at 40× magnification with a phase contrast optical microscope, of untreated hair;

FIG. 2b shows images, acquired at 40× magnification with a phase contrast optical microscope, of hair treated with a composition (lotion) containing 0.2% of methylspermidine free base according to Example 6;

FIG. 2c shows images, acquired at 40× magnification with a phase contrast optical microscope, of hair treated with a PLACEBO lotion according to Example 6.

FIG. 3 illustrates a slide on which a fraction of hair divided into 4 parts of about 2-3 mm according to the method of Example 6 is laid.

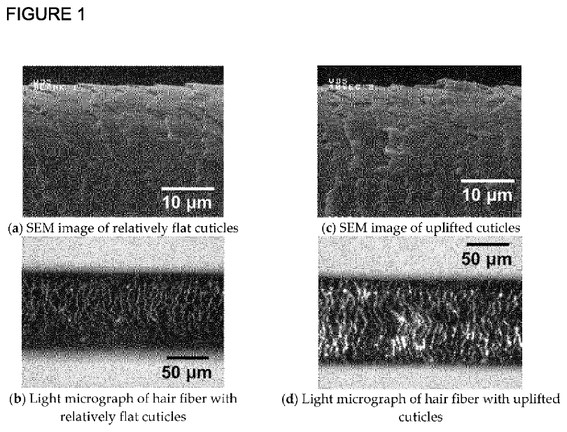

### DETAILED DESCRIPTION OF THE INVENTION

The present invention originates from having found that the application of a compound of formula (I), or a composition containing it, on an external hair structural component, such as the cuticle, reduces breakage or fracture points in the hair, determining a combined restructuring and aesthetic effect.

The effect of the compounds of formula (I) and of the composition containing them is limited to the external keratin structure of the hair, which is typically based on stratified keratin scales.

Hair treatment with a compound of formula (I), or a cosmetic composition containing it, increases hair softness and shine, thus improving its outward appearance.

According to one aspect, the invention relates to the use of a compound of formula (I) as defined in claim 1.

For the cosmetic uses of the invention, a preferred compound of formula (I) is N¹-methylspermidine, i.e. N-(3-aminopropyl)-N¹-methyl-1,4-butanediamine (CAS Registry Number 51460-23-2), of formula:

H₂N—(CH₂)₃—N¹(CH₃)—(CH₂)₄—NH₂ (Ia)

in the form of a free base or a cosmetically acceptable salt, for example trichlorohydrate (3HCl) or trimaleate.

A further preferred compound of formula (I) according to the present invention is N¹-cyclohexylspermidine, i.e. N-(3-aminopropyl)-N¹-cyclohexyl-1,4-butanediamine (CAS Registry Number 183070-28-2), having the formula:

H₂N—(CH₂)₃—N¹(C₆H₁₁)—(CH₂)₄—NH₂ (Ib)

A further preferred compound of formula (I) according to the present invention is N¹-ethylspermidine, i.e. N-(3-aminopropyl)-N¹-ethyl-1,4-butanediamine (CAS Registry Number 97141-36-1) of formula:

H₂N—(CH₂)₃—N¹(C₂H₅)—(CH₂)₄—NH₂ (Ic)

A further preferred compound of formula (I) according to the present invention is N¹-propylspermidine, i.e. N-(3-aminopropyl)-N¹-propyl-1,4-butanediamine (CAS Registry Number 62659-14-7), having the formula:

H₂N—(CH₂)₃—N¹(C₃H₇)—(CH₂)₄—NH₂ (Id)

A further preferred compound of formula (I) according to the present invention is N¹-isobutylspermidine, i.e. N-(3-aminopropyl)-N¹-isobutyl-1,4-butanediamine, of formula:

H₂N—(CH₂)₃—N¹(C₄H₉)—(CH₂)₄—NH₂ (Ie)

According to one aspect, the invention relates to the cosmetic use of a cosmetic composition containing alkyl spermidines as defined herein, in particular methylspermidine, by applying it on hair shafts for restructuring the cuticle and/or improving the outward appearance thereof.

Advantageously, the composition is applied to the hair shafts, to the cuticular portion/component which typically comprises 6-10 layers of superimposed keratin flakes/scales, arranged in a weaving pattern.

In one embodiment, the composition applied to the hair shafts is removed by rinsing with water, optionally with a detergent such as a shampoo.

According to one aspect, the invention therefore relates to the non-therapeutic, cosmetic use of a composition as defined in the appended claim 8.

The composition described herein is for external use, in particular for application to the outer cuticle layer of hair.

The cosmetic use according to the invention results in a redensification of the hair fiber, an improvement of the mechanical properties of the hair fiber, an increase in resistance to breakage, a reduction in hair porosity. These effects reduce the effects of humidity and the aggressions of external agents that can penetrate and damage the inner structures.

Furthermore, the application of the composition described herein on hair favours the cuticles closure, increases combability and reduces the mechanical stress to which hair is subjected during typical grooming operations.

In some embodiments, the composition of the invention contains a compound of formula (I) in an amount ranging from 0.0001 to 10% by weight, from 0.01 to 5% by weight, or from 0.1 to 2% by weight by weight.

Advantageously, the compound of formula (I), or a cosmetic composition containing it, is applied in a cosmetically effective amount to the external structure of the hair, typically on cuticles.

Preferably the cosmetic composition is applied to at least a portion of the hair shaft and not rinsed off (e.g. restructuring lotion) or left to act for a period of time sufficient to achieve a cosmetic effect, for example for 1-60 minutes, 5-40 minutes, preferably 8-20 minutes.

For example, the composition may be applied before washing the hair with a cleansing product such as a shampoo.

For example, the cosmetic treatment method for hair provides for the application on dry hair of a cosmetically effective amount of the composition described herein, for example 1-5 mL in the case of a hair lotion or 1 or 2 knobs of composition in the case of a balm or mask. After being applied along the entire hair length, the composition is left in place for at least 10 minutes. Subsequently, the composition is removed by rinsing with water, optionally followed by washing with a hair shampoo.

In some embodiments, the composition of the invention comprises further cosmetically active substances.

In its multiple embodiments, the composition incorporates one or more substances beneficial to the hair selected from hair conditioners, emollients, hair lubricating agents, protein hydrolysates, amino acids, moisturizing agents for scalp hygiene, substances for protection from sun rays such as proper sunscreens and substances that absorb in the UV and visible spectrum (blue light), volumizing agents, conditioners, fixing resins for hair styling. Other substances such as: solvents (ethanol, isopropyl alcohol, in addition to water), viscosifiers, film-forming agents, hydrotropes, rheological modifiers, chelating or sequestering agents, tensolites or surfactants, opacifying or pearlescent substances, emulsifiers, perfumed substances, natural and non-natural dyes, antioxidants, stabilizers, humectants, preservatives and substances with antimicrobial action; may be part of the composition.

According to some embodiments, the cosmetic composition comprises a further cosmetically active ingredient based on a hydrogenated castor oil/sebacic acid copolymer. This active ingredient combined with a compound of formula (I), in particular methylspermidine, exerts a synergistic restructuring effect on the hair fiber. This combination works by restructuring it from the inside, acting on the uplifted cuticles and sealing them, polishing the hair shaft. Treated hair appears shinier and less susceptible to external aggressions.

According to some embodiments, the cosmetic composition described herein comprises a compound of formula (I), such as methylspermidine, formulated with hyaluronic acid, for example hydroxypropyltrimonium hyaluronate, and/or hydrogenated castor oil/sebacic acid copolymer.

This combination of cosmetically active ingredients has a restructuring effect on the hair keratin, a cementing effect on the uplifted hair cuticles, and filming and moisturizing effect.

The combination of the three cosmetically active ingredients referred to above repairs the hair breakage points, which points are the most fragile and susceptible to external aggressions, and form a protective film that balances the physiological electric charge of the hair and favour the closure of the external cuticle cells.

Hair treated with the composition according to any of the embodiments described herein appears shiny and less susceptible to the action of moisture and dehydration.

The cosmetic composition described herein may be in solid, semi-solid, fluid, or semi-fluid form.

Suitable formulations include creams, gels, pomades, pastes, ointments, masks, emulsions, lotions, solutions, aerosols.

Preferably the composition is in the form of a mask to be applied on a keratin structure of the human body.

In the case of formulations in fluid or semi-fluid form, the cosmetically active ingredients and/or any excipients may be diluted in a carrier in a physiologically acceptable fluid form, such as water or other liquids, suitable for local application.

By way of example, the composition in fluid or liquid form may be a spray composition to be applied by spraying it onto the hair shaft. The composition in the form of a spray may be dispensed into a pressurized aerosol container, optionally containing a propellant to force the composition out of the container.

Within the scope of the invention, the term keratin or keratin-based structure means hair, eyelashes or eyebrows of an individual.

The term individual refers to a mammal, typically a human being or a pet, such as a dog or cat.

The term “cosmetically acceptable” as used herein means that the cosmetic composition or its active components are suitable for general application to the skin and when applied do not cause undesirable toxicity, allergic response, redness, incompatibility, instability and similar reactions.

The following examples are provided for purposes of illustration of the present invention.

### Example 1

TREATMENT MASK FOR DAMAGED HAIR

| | Amount w/v |
|---|---|
| Ingredient (INCI) | (%) |
| Aqua | q.s. to 100 mL |
| Ammonium Acryloyldimethyltaurate/ | 0.50-2.00 |
| VP Copolymer | |
| Peg-40 Hydrogenated castor oil | 0.20-3.00 |
| Parfum | 0.10-1.00 |
| Potassium sorbate | 0.04-0.49 |
| Phenoxyethanol | 0.10-1.00 |
| Panthenol | 0.10-0.50 |
| Glycerin | 0.50-5.00 |
| Sorbitol | 0.10-1.00 |
| Linum usitatissimum (Linseed) seed oil | 0.10-2.00 |
| Methylspermidine (free base) | 0.010-0.30 |
| Tetrasodium glutamate diacetate | 0.025-0.10 |
| Citric acid | 0.05-0.90 |
| Sodium hydroxide | 0.01-0.50 |
| Polyquaternium-64 | 0.20-0.25 |
| Polyquaternium-11 | 0.05-0.50 |

### Example 2

CONDITIONING BALM

| | Amount w/v |
|---|---|
| Ingredient (INCI) | (%) |
| Aqua | q.s. to 100 mL |
| Disodium EDTA | 0.025-0.05 |
| Panthenol | 0.50-1.50 |
| Oryza Sativa (Rice) Bran Extract | 0.050-0.20 |
| Butylene glycol | 0.055-3.00 |
| Hydroxyethyl cellulose | 0.10-0.90 |
| Cetrimonium chloride | 0.50-5.00 |
| Bis-Isobutyl PEG/PPG-20/35/ | 0.05-0.75 |
| Amodimethicone Copolymer | |
| Cetyl Ethylhexanoate | 0.05-0.40 |
| Polysorbate 80 | 0.05-0.40 |
| C12-13 alkyl lactate | 0.50-5.00 |
| Glyceryl stearate | 1.00-6.00 |
| PEG-100 stearate | 0.50-4.00 |
| Citric acid | 0.05-1.00 |
| Sodium hydroxide | 0.01-0.50 |
| Dimethicone | 1.00-6.00 |
| Dimethiconol | 0.10-1.00 |
| Cetearyl alcohol | 1.00-7.00 |
| Limnanthes Alba (Meadowfoam) Seed Oil | 0.050-1.00 |
| Potassium sorbate | 0.04-0.49 |
| Sodium benzoate | 0.04-0.35 |
| Phenoxyethanol | 0.10-1.00 |
| Ethylhexylglycerin | 0.01-0.25 |
| Methylspermidine (free base) | 0.010-0.25 |
| Parfum | 0.10-0.30 |

### Example 3

POST-DYEING TREATMENT

| | Amount w/v |
|---|---|
| Ingredient (INCI) | (%) |
| Aqua | q.s. to 100 mL |
| Peg-40 Hydrogenated castor oil | 0.50-2.00 |
| Alcohol denat | 15.0-20.0 |
| Panthenol | 0.10-0.50 |
| Methylspermidine (free base) | 0.010-0.40 |
| Disodium EDTA | 0.025-0.05 |
| Parfum | 0.10-0.40 |
| Ceramide III | 0.001-0.25 |
| Peg-40 Hydrogenated castor oil | 0.30-2.00 |
| Lactic acid | q.s. to pH 4.5 |

### Example 4

RESTRUCTURING LOTION

| | Amount w/v |
|---|---|
| Ingredient (INCI) | (%) |
| Aqua | q.s. to 100 mL |
| Alcohol denat | 15.0-20.0 |
| Methylspermidine (free base) | 0.005-0.30 |
| Biotin | 0.01-0.10 |
| Calcium pantothenate | 0.1-3.0 |
| PEG-40 Hydrogenated Castor Oil | 0.5-2.0 |
| Parfum | 0.20 |
| Lactic acid | q.s. to pH 5.0 |

### Example 5

RESTRUCTURING SHAMPOO

| | Amount w/w |
|---|---|
| Ingredient (INCI) | (%) |
| Sodium laurylglucosides hydroxypropylsulfonate | 1.00-5.00 |
| Magnesium laureth sulfate | 5.00-9.00 |
| Sodium cocoamphoacetate | 0.05-3.00 |
| Olive Oil PEG-7 Esters | 0.50-1.00 |
| Peg-200 Hydrogenated Glyceryl Palmate | 0.10-2.00 |
| Polyquaternium-10 | 0.10-0.50 |
| PEG-120 Methyl glucose dioleate | 0.10-2.00 |
| Sodium lauroyl sarcosinate | 1.00-4.00 |
| Tetrasodium EDTA | 0.05-0.20 |
| Methylspermidine (free base) | 0.001-0.21 |
| Panthenol | 0.01-3.0 |
| Phytantriol | 0.01-0.10 |
| Parfum | 0.10-0.80 |
| Glycol Distearate | 0.50-1.00 |
| Cocamidopropyl Betaine | 0.01-5.00 |
| Potassium sorbate | 0.04-0.49 |
| Sodium benzoate | 0.04-0.35 |
| Sodium hydroxide | 0.001-0.20 |
| Sodium chloride | 0.001-1.00 |
| Citric acid | q.s. pH 5.5 |
| Aqua | q.s. 100.00 |

### Example 6

### Experimental Tests of Hair Breakage

### Equipment Used

Quadritriocular phase contrast microscope (Mod: DMIL);
Dig Camera DFC450c;
Leica Application Suite LASX v. 3.0.0.15697.

### Methods

Methylspermidine free base (0.2%) was applied on to 10 hair strands for 10 minutes and then rinsed off.

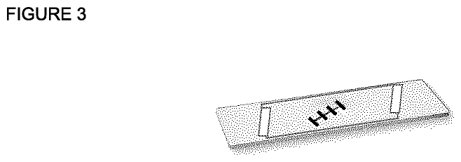

Untreated hair strands were used as a NEGATIVE CONTROL.
a) A fragment of about 1 cm was obtained from each hair (3 hairs per strand);
b) The fragment obtained was placed on a microscope slide and dry mounted with a coverslip and transparent adhesive tape (Ajda et al., 2011; Dhurat et al., 2009);
c) With the aid of a felt-tip pen, each fragment was divided into 4 parts of about 2-3 mm in size (FIG. 3);

Using an optical, fluo-trinocular, inverted phase microscope, at 40× magnification, and the LasX Core™ software, images of the three different portions obtained by dividing each fragment were acquired;

A general, visual assessment of the state of the hair (open/exposed cuticles) and numerical assessment were carried out using the ImageJ image processing software.

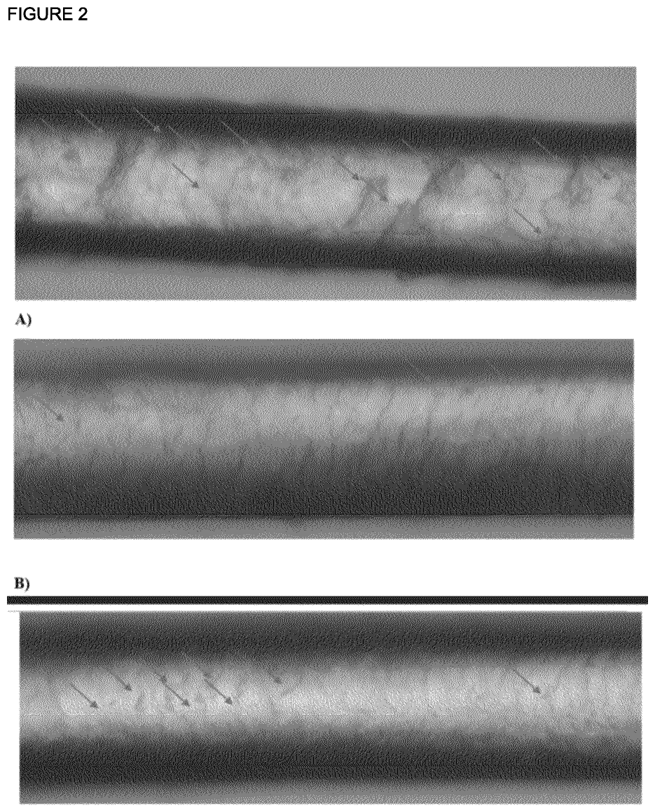

The enclosed FIG. 2 illustrates representative images of micrographs of untreated hair (a), hair treated with restructuring lotion (b) and treated with PLACEBO lotion (c). Images were acquired at 40× magnification with a phase contrast microscope.

| | Breakage/fracture |
|---|---|
| | points |
| Untreated hair | 17.25 ± 3.32 |
| Hair treated with RESTRUCTURING | 6.45 ± 2.76 |
| LOTION OF EXAMPLE 4 | |
| Hair treated with PLACEBO | 14.67 ± 2.81 |

Treatment of strands with 0.2% methylspermidine (RESTRUCTURING LOTION) reduces breakage/fracture points in the hair shaft by 63%.

RESTRUCTURING LOTION (test)

| | Amount w/v |
|---|---|
| Ingredient (INCI) | (%) |
| Aqua | q.s. to 100 mL |
| Alcohol denat | 20.0 |
| Methylspermidine (free base) | 0.20 |
| Lactic acid | q.s. to pH 4.6 |

PLACEBO

| | Amount w/v |
|---|---|
| Ingredient (INCI) | (%) |
| Aqua. | q.s. to 100 mL |
| Alcohol denat | 20.0 |
| Lactic acid | q.s. to pH 4.6 |

1. A cosmetic method for restructuring the cuticle of a keratin-based structure of an individual and improve its outward appearance, said method comprising the step of applying a compound of formula (I)
H₂N—(CH₂)₃—N¹(R)—(CH₂)₄—NH₂, (I)
wherein R is a substituent, selected from
linear or branched, saturated alkyl, having from 1 to 6 carbon atoms;
saturated cycloalkyl group, preferably cyclohexyl,
in the form of a free base or a pharmaceutically acceptable salt thereof,
to at least a portion of the cuticle of said keratin-based structure.

2. The cosmetic method according to claim 1, wherein said compound of formula (I) is N¹-methylspermidine of formula
H₂N—(CH₂)₃—N¹(CH₃)—(CH₂)₄—NH₂. (Ia)

3. The cosmetic method according to claim 1, wherein said compound of formula (I) is N¹-cyclohexylspermidine, or N-(3-aminopropyl)-N¹-cyclohexyl-1,4-butanediamine of formula:
H₂N—(CH₂)₃—N¹(C₆H₁₁)—(CH₂)₄—NH₂, (Ib)

4. The cosmetic method according to claim 1, wherein said compound of formula (I) is N¹-ethylspermidine, or N-(3-aminopropyl)-N¹-ethyl-1,4-butanediamine of formula:
H₂N—(CH₂)₃—N¹(C₂H₅)—(CH₂)₄—NH₂. (Ic)

5. The cosmetic method according to claim 1, wherein said compound of formula (I) is N¹-propylspermidine, or N-(3-aminopropyl)-N¹-propyl-1,4-butanediamine of formula:
H₂N—(CH₂)₃—N¹(C₃H₇)—(CH₂)₄—NH₂. (Id)

6. The cosmetic method according to claim 1, wherein said compound of formula (I) is N¹-isobutylspermidine, or N-(3-aminopropyl)-N¹-isobutyl-1,4-butanediamine of formula:
H₂N—(CH₂)₃—N¹(C₄H₉)—(CH₂)₄—NH₂. (Ie)

7. The cosmetic method according to claim 1, wherein said keratin-based structure is hair, eyelashes or eyebrows.

8. A cosmetic method for restructuring the cuticle and/or improving the outward appearance of a keratin structure comprising the step of applying on the shaft of the keratin structure a composition comprising a compound of formula (I) according to claim 1 and a cosmetically acceptable carrier.

9. A cosmetic method carrier for treating the lifting of overlapping keratin scales of hair, eyelashes, and/or eyebrows and recompacting them, said method comprising the step of applying on the keratin scales of said hair, eyelashes, and/or eyebrows of a composition comprising a compound of formula (I) according to claim 1 and a cosmetically acceptable carrier.

10. A cosmetic method for reducing the breakage points in the shaft of hair, eyelashes and/or eyebrows, and increasing their resistance to breakage comprising the step of applying a composition on the keratin scales of said hair, eyelashes, and/or eyebrows, said composition comprising a compound of formula (I) according to claim 1 and a cosmetically acceptable carrier.

11. The cosmetic method according to claim 8, wherein the application of said composition to the scalp is substantially excluded.

12. The method according to claim 8, wherein the composition is a lotion, solution, gel, cream, paste, emulsion, hair mask.

13. The method according to claim 9, wherein said solution is an aqueous or hydroalcoholic solution.

14. The cosmetic method according to claim 10, wherein the application of said composition to the scalp is substantially excluded.
